(19) United States
(12) Patent Application Publication
(10) Pub. No.: US 2025/0186515 A1
(43) Pub. Date: Jun. 12, 2025

(54) PROBIOTIC COMPOSITIONS AND METHODS

(71) Applicant: UNIVERSITY OF OTTAWA, Ottawa (CA)

(72) Inventors: Chantal MATAR, Ottawa (CA); Nour YAHFOUFI, Manotick, Ontario (CA); Nafissa ISMAIL, Ottawa (CA); Jean-Francois MALLET, Gatineau (CA)

(73) Assignee: UNIVERSITY OF OTTAWA, Ottawa (CA)

(21) Appl. No.: 18/978,215

(22) Filed: Dec. 12, 2024

Related U.S. Application Data
(62) Division of application No. 17/769,301, filed on Apr. 14, 2022.
(60) Provisional application No. 62/916,921, filed on Oct. 18, 2019.

(51) Int. Cl. A61K 35/741; A61K 9/00; A61K 35/00; A61K 35/744; A61K 35/745; A61K 35/747; A61K 36/06; A61P 1/00; A61P 37/02

(52) U.S. Cl. CPC A61K 35/741; A61K 9/0053; A61K 35/744; A61K 35/745; A61K 35/747; A61K 36/06; A61P 1/00; A61P 37/02; A61K 2035/115

(57) ABSTRACT

Provided herein are probiotic compositions comprising bacteria of the genus Rouxiella and in particular, Rouxiella badensis. Also provided are methods of using these probiotic compositions for promoting gut health including improving intestinal barrier function and to maintain or regulate intestinal homeostasis.

### FIELD OF THE INVENTION

This invention pertains generally to probiotic compositions and methods and, more particularly to probiotic compositions comprising bacteria of the genus Rouxiella and more specifically probiotic compositions comprising Rouxiella badensis or a subspecies or a strain thereof and methods of using these probiotic compositions.

### BACKGROUND OF THE INVENTION

The intestinal microbiota comprises more than 100 billon of microorganisms and more than 1000 different bacterial species that have an important role in promoting health (Qin J., et al. 2010. Nature, 464:59-65. doi: 10.1038/nature08821). Microbiota prevents pathogen colonization and maintains the gut mucosal immunity.

Probiotics are defined by the World Health Organization as live microorganisms that, “when administered in adequate amounts, confer a health benefit on the host.” The health benefits which have been claimed for probiotics include improvement of the normal microbiota, immunomodulatory effects, prevention of the infectious diseases reduction of serum cholesterol, anticarcinogenic activity, immune adjuvant properties, alleviation of intestinal bowel disease (IBD) symptoms and improvement in the digestion of lactose in intolerant hosts (Kumar M V V, et al. British Journal of Nutrition. 2012; 107(7): p. 1006; Fábrega M J et al. Front Microbiol 2017; 11;8:1274. doi: 10.3389/fmicb.2017.01274; Velez E M, et al. Br J Nutr 2015; 114(4): 566-76. doi: 10.1017/S0007114515001981; Nelson H S. Allergy Asthma Proc 2016; 37(4): 268-72. doi: 10.2500/aap.2016.37.3966 and He J, et al. Medicine (Baltimore) 2017; 96(51): e9166. doi:10.1097/MD.0000000000009166.)). There is evidence suggesting probiotic bacteria can reduce intestinal colonization by Salmonella typhimurium (Deriu, E. et al. Cell Host Microbe 2013, 14(1): 26-37. Doi: 10.1016/j.chom.2013.06.007)

The genera most commonly used for probiotic preparations are Lactobacillus, Bifidobacterium, Streptococcus, Lactococcus and some fungal strains. Foods containing probiotic microorganisms for human consumption include fermented milks, cheeses, fruit juices, wine and sausages amongst others. Mixed cultures of live microorganisms are also used in probiotic preparations.

This background information is provided for the purpose of making known information believed by the applicant to be of possible relevance to the present invention. No admission is necessarily intended, nor should be construed, that any of the preceding information constitutes prior art against the present invention.

### SUMMARY OF THE INVENTION

An object of the present invention is to provide probiotic compositions and methods. In accordance with an aspect of the present invention, there is provided an oral probiotic composition comprising Rouxiella sp., optionally Rouxiella badensis and in some embodiments a subspecies or a strain thereof.

In accordance with another aspect of the invention, there is provided a food product comprising an effective amount of a probiotic bacteria of the genus Rouxiella, optionally the species Rouxiella badensis and in some embodiments a subspecies or strain thereof.

In accordance with another aspect of the invention, there is provided a method of promoting gut mucosal immunity in a mammal, the method comprising orally administering an isolated Rouxiella sp., a probiotic composition comprising Rouxiella sp. or a food product comprising Rouxiella sp., optionally Rouxiella badensis and in some embodiments a subspecies or strain thereof.

In accordance with another aspect of the invention, there is provided a method of restoring and/or increasing the intestinal epithelial barrier, the method comprising orally administering an isolated Rouxiella sp., a probiotic composition comprising Rouxiella sp. or a food product comprising Rouxiella sp., optionally Rouxiella badensis and in some embodiments a subspecies or strain thereof.

In accordance with another aspect of the invention, there is provided a method of protecting against Salmonella infection, the method comprising orally administering an isolated Rouxiella sp., a probiotic composition comprising Rouxiella sp. or a food product comprising Rouxiella sp, optionally Rouxiella badensis and in some embodiments a subspecies or strain thereof.

### BRIEF DESCRIPTION OF THE DRAWINGS

These and other features of the invention will become more apparent in the following detailed description in which reference is made to the appended drawings.

FIG. 1 illustrates neighbor-joining unrooted tree based on rrs gene sequences. Numbers indicate substitutions per nucleotide position.

FIG. 2 illustrates neighbor-joining unrooted tree based on MLSA. Numbers indicated substitutions per nucleotide position.

FIG. 3 illustrates the prophage positions in Rouxiella badensis acadiensis chromosome.

FIG. 4 shows colony morphology of Rouxiella badensis acadiensis plated on SDA and TSA media.

FIG. 5 shows resistance of Rouxiella badensis acadiensis to extreme conditions. (A) After 2 and 4 h of the bacterium incubation in TSA broth pH: 2, 3, 4, 5 and 7 at 30° C., serial dilutions were made and plate in agar plate for the CFU determination. (B) Growth of Rouxiella badensis acadiensis at 30° C. in TSA broth containing 0.3, 0.5 and 1% w/v bile salts for 2 and 4 h. Results were expressed as the Log10 of the Mean±SEM of three independent experiments.

FIG. 6 shows Rouxiella badensis acadiensis did not cause any toxicity effect on mammalian cells. Mice peritoneal macrophages were incubated at 37° C., 5% CO₂ for 24 h in presence of (A) increasing concentration Rouxiella badensis acadiensis (10⁷ to 10¹⁰ CFU/ml) and (B) the supernatants of a 18 h bacterium culture. Cell viability was determined by the MTT method and was expressed as the ratio between viable cells in the presence and absence of the compound multiplied by 100. Bars represent the mean±SEM of three experiments carried out in duplicate.

FIG. 7 shows bacterial composition at phylum level (10 most abundant phyla) following treatment with 10⁹ CFU/ml of Rouxiella badensis acadiensis daily for three weeks, prebiotic Protocatechuic acid (PCA), 100 mg/kgBW, both Rouxiella badensis acadiensis and prebiotic.

FIG. 8 shows two taxa with significantly different abundance across groups were found and linked to the administration of prebiotic or Rouxiella badensis acadiensis.

FIG. 9 shows adherence of Rouxiella badensis acadiensis to the intestinal epithelium. PBS (control), or Rouxiella badensis acadiensis (10⁹ CFU/ml) were administered by intragastric inoculation to Balb/c mice. Five and 15 minutes later mice were killed and their small intestine removed for (A) scanning electron microscopy, and (B) transmission electron microscopy, to evaluate the bacteria adhesion to the epithelium. Panels: I and IV-Control, II, V and III, VI: 5 and 15 minutes after Rouxiella badensis acadiensis administration, respectively.

FIG. 10 shows micrographs of small intestine sections. Animals were fed with (A) conventional diet (control) or (B and C) Rouxiella badensis acadiensis (10⁹ CFU/ml) for 7 days and three months, respectively, in their beverages. Small intestines were removed and tissue sections were stained with hematoxylin and eosin at the end of the feed periods. Red arrows indicate Paneth cells while black arrows denote Globet cells. (D) Transmission electron microscopy, showing Goblet cells in Rouxiella badensis acadiensis fed mice.

FIG. 11 shows effect of Rouxiella badensis acadiensis on intestinal integrity and prevention of LPS Induced-Inflammation. (A) shows micrographs of hematoxylin-eosin stained sections of small intestine of control group. (B) shows micrograph of hematoxylin-eosin stained sections of small intestine of LPS injected group. (C) shows micrographs of hematoxylin-eosin stained sections of small intestine of probiotic treated control group. (D) shows micrograph of hematoxylin-eosin stained sections of small intestine of LPS injected, probiotic treated group.

FIG. 12 shows phagocytic activity of peritoneal macrophages. Animals were fed with conventional diet (control) or 10⁹ CFU/ml of Rouxiella badensis acadiensis for (A) 7 days and (B) three months, respectively. The phagocytosis of macrophages was performed using an opsonized Saccharomyces cerevisiae suspension. Percentages of phagocytosis were expressed as the percentage of phagocyting macrophages in 100 cells counted in an optical microscope. Results are expressed as the Mean±SEM of three experiments.

FIG. 13 shows a determination of the antimicrobial activity in animals' intestinal fluids. S. aureus and S. typhimirium (10⁹ CFU/ml) were incubated for 2 h at 37° C. in the presence of the intestinal fluids of mice fed with conventional diet, or Rouxiella badensis acadiensis for consecutive (A and B) seven days, (C and D) 1 month, or (D and E) 3 month, respectively. After the co-incubation, viable bacteria were determined by plate counts agar. Results are expressed as CFU/ml. **p<0.01, ***p<0.001.

FIG. 14 shows influence of Rouxiella badensis acadiensis on the body weight. Animals were fed with conventional diet (control) or diet supplemented with 10⁹ CFU/ml of Rouxiella badensis acadiensis for 1 or 3 months consecutive respectively. Body weight was determined every 2 or 4 days. Results were expressed as the % of mean of the initial weight (weight registered the day before bacterium administration).

FIG. 15 shows results of challenge with S. Typhimurium in Rouxiella badensis acadiensis fed mice. Mice fed with conventional diet or Rouxiella badensis acadiensis supplemented diet for seven days were orally infected with S. Typhimurium. After the challenge one groups of mice also received Rouxiella badensis acadiensis supplemented diet for seven days following challenge (continuous). (A) Translocation of bacteria to liver and spleen in mice 7 days' post infection. (B) Survival of the animals to Salmonella challenge.

FIG. 16 shows mean±SEM of concentration of different (A) anti-inflammatory cytokines IL-10, (B) pro-inflammatory cytokine-IL-6 as well a (c) IgA in the intestinal fluid (CTR) control mice fed 1% sucrose or Rouxiella badensis acadiensis treated group 10⁸ CFU/mouse/day for 7 days. Number of animals per group is n=10. Difference is considered significant between groups if *p<0.05 ns=non-significant difference when p>0.05.

FIG. 17 shows mean±SEM of the number of IgA, IgG and IL10 positive cells populations in 10 fields of objective 100× in the ileum of mice fed 1% sucrose (CTR) or Rouxiella badensis acadiensis (labelled AV) fed Rouxiella badensis acadiensis 10⁸ CFU/mouse/day for 7 days. Significant difference between mice if *p<0.05, ** if p<0.01 and *** if p<0.001.

FIG. 18 shows mean±SEM of relative expression of miR145 and miR146a in the brain and ileum of (CTR) control mice fed 1% sucrose or Rouxiella badensis acadiensis treated group 10\hat{ }8 CFU/mouse/day for 7 days. Significant difference between mice exists if *p<0.05.

FIG. 19 shows intestinal permeability measured by FITC-dextran in serum of animals that received conventional diet (control), or the supplementation with 10⁹ Rouxiella badensis acadiensis.

FIG. 20 shows cadherin in jejunum. Animals received conventional diet (control/top), or the supplementation with Rouxiella badensis acadiensis (bottom). Magnification: 40×.

FIG. 21 shows occludin in ileum. Animals received conventional diet (control/top), or the supplementation with Rouxiella badensis acadiensis (bottom). Magnification: 40×.

FIG. 22 shows production of antimicrobial peptides in the intestinal fluids of animals that received conventional diet (control/top), or the supplementation with Rouxiella badensis acadiensis (RBA).

### DETAILED DESCRIPTION OF THE INVENTION

The present invention relates to probiotic bacteria belonging to the genus Rouxiella, to compositions comprising the same and methods of using the probiotic strains and compositions.

### Definitions

Unless defined otherwise, all technical and scientific terms used herein have the same meaning as commonly understood by one of ordinary skill in the art to which this invention pertains.

The term “probiotic” means live microorganisms that, when administered in adequate amounts, confer a health benefit on the host.

The term “microbiota” means the ecological community of microorganisms found in and on a multicellular organism and includes commensal, symbiotic and pathogenic microorganisms.

The term “intestinal microbiota” means microbiota within the intestines.

The term “Rouxiella badensis—like probiotic bacteria” or “RBL probiotic bacteria” refers to the probiotic bacteria characterized in Example 1 and having has a rrs gene sequence as set forth in SEQ ID NO:1, has a groL gene sequence as set forth in SEQ ID NO:2, a gyrB gene sequence as set forth in SEQ ID NO:3 a fusA gene sequence at as set forth in SEQ ID NO:4, has a pyrG gene sequence as set forth in SEQ ID NO:5, a rplB gene sequence as set forth in SEQ ID NO:6, a rpoB gene sequence as set forth in SEQ ID NO:7 and a sucA gene sequence as set forth in SEQ ID NO:8.

The abbreviation “CFU” is short for colony forming unit and refers to the amount of bacteria in a probiotic that are viable and capable of dividing and forming colonies

### Probiotic Bacteria:

Probiotic bacteria closely related to Rouxiella badensis and in particular, closely related to strains 421 or 323 of this species. Optionally, the invention comprises the probiotic bacteria isolated from the microflora of lowbush blueberry (Vaccinium angustifolium) and referred herein as to Rouxiella badensis acadiensis and deposited with ATTC® Patent Depository (10801 University Boulevard Manassas, Virginia 20110-2209) in its capacity as an International Depository Authority on Jul. 22, 2020 under Accession Number PTA-126681.

In other embodiments, the invention comprises Rouxiella badensis, optionally strains 421 or 323.

In other embodiments, the invention comprises a probiotic composition comprising at least one, at least two, or at least three probiotic bacteria belonging to the genus Rouxiella and optionally one or more probiotic bacteria belonging to different genus or probiotic yeast strains. In some embodiments, one or more probiotic bacteria belonging to different genus are selected from the group consisting of Lactobacillus, Bifidobacterium, Streptococcus, and Lactococcus.

The probiotic bacteria of the invention can be defined by reference to specific gene sequences. In one embodiment, the probiotic bacteria has a rrs gene sequence at least 97% identical to the sequence set forth in SEQ ID NO:1, has a groL gene sequence at least 97% identical to the sequence set forth in SEQ ID NO:2 and a gyrB gene sequence at least 97% identical to the sequence set forth in SEQ ID NO:3.

In one embodiment, the probiotic bacteria has a rrs gene sequence at least 97.5% identical to the sequence set forth in SEQ ID NO:1, has a grol gene sequence at least 97.5% identical to the sequence set forth in SEQ ID NO:2 and a gyrB gene sequence at least 97.5% identical to the sequence set forth in SEQ ID NO:3.

In one embodiment, the probiotic bacteria has a rrs gene sequence at least 98.5% identical to the sequence set forth in SEQ ID NO:1, has a groL gene sequence at least 98.5% identical to the sequence set forth in SEQ ID NO:2 and a gyrB gene sequence at least 98.5% identical to the sequence set forth in SEQ ID NO:3.

In one embodiment, the probiotic bacteria has a rrs gene sequence at least 99.9% identical to the sequence set forth in SEQ ID NO:1, has a groL gene sequence at least 99.9% identical to the sequence set forth in SEQ ID NO:2 and a gyrB gene sequence at least 99.8% identical to the sequence set forth in SEQ ID NO:3.

In one embodiment, the probiotic bacteria has a rrs gene sequence as set forth in SEQ ID NO:1, has a groL gene sequence as set forth in SEQ ID NO:2 and a gyrB gene sequence as set forth in SEQ ID NO:3.

In one embodiments, the invention comprises a probiotic bacteria has a fusA gene sequence at least 97% identical to the sequence set forth in SEQ ID NO:4, has a pyrG gene sequence at least 97% identical to the sequence set forth in SEQ ID NO:5, a rplB gene sequence at least 97% identical to the sequence set forth in SEQ ID NO:6, a rpoB gene sequence at least 97% identical to the sequence set forth in SEQ ID NO:7 and a sucA gene sequence at least 97% identical to a sequence set forth in SEQ ID NO:8.

In one embodiments, the invention comprises a probiotic bacteria has a fusA gene sequence at least 97.5% identical to the sequence set forth in SEQ ID NO:4, has a pyrG gene sequence at least 97.5% identical to the sequence set forth in SEQ ID NO:5, a rplB gene sequence at least 97.5% identical to the sequence set forth in SEQ ID NO:6, a rpoB gene sequence at least 97.5% identical to the sequence set forth in SEQ ID NO:7 and a sucA gene sequence at least 97.5% identical to a sequence set forth in SEQ ID NO:8.

In one embodiment, the invention comprises a probiotic bacteria has a fusA gene sequence at 100% identical to the sequence set forth in SEQ ID NO:4, has a pyrG gene sequence 100% identical to the sequence set forth in SEQ ID NO:5, a rplB gene sequence 100% identical to the sequence set forth in SEQ ID NO:6, a rpoB gene sequence at least 99.8% identical to the sequence set forth in SEQ ID NO:7 and a sucA gene sequence at least 99.8% identical to a sequence set forth in SEQ ID NO:8.

In one embodiment, the invention comprises a probiotic bacteria has a fusA gene sequence at as set forth in SEQ ID NO:4, has a pyrG gene sequence as set forth in SEQ ID NO:5, a rplB gene sequence as set forth in SEQ ID NO:6, a rpoB gene sequence as set forth in SEQ ID NO:7 and a sucA gene sequence as set forth in SEQ ID NO:8.

In one embodiment, the probiotic bacteria of the invention has a rrs gene sequence as set forth in SEQ ID NO:1, has a groL gene sequence as set forth in SEQ ID NO:2, a gyrB gene sequence as set forth in SEQ ID NO:3 a fusA gene sequence at as set forth in SEQ ID NO:4, has a pyrG gene sequence as set forth in SEQ ID NO:5, a rplB gene sequence as set forth in SEQ ID NO:6, a rpoB gene sequence as set forth in SEQ ID NO:7 and a sucA gene sequence as set forth in SEQ ID NO:8.

In some embodiments, the probiotic bacteria chromosome includes intact prophage that shares sequences identity to the temperate Salmonella SN5 phage.

In some embodiments, the probiotic bacteria has a plasmid having a sequence as set forth in SEQ ID NO:9 or SEQ ID NO:10.

In some embodiments, the probiotic bacteria has a first plasmid having a sequence as set forth in SEQ ID NO:9 and a second plasmid having a sequence as set forth in SEQ ID NO:10.

In one embodiment, the probiotic bacteria is a Rouxiella badensis—like probiotic bacteria, optionally Rouxiella badensis or a subspecies or strain thereof. In some embodiments, the probiotic bacteria is a bacteria substantially identical to the bacteria deposited with the ATTC® Patent Depository under Accession Number PTA-126681 and referred to as Rouxiella badensis acadiensis.

In some embodiments, the probiotic bacteria comprise bacteriocins, ribosomally synthesized antibacterial peptides (colicin V and Bacteriocin production cluster) and/or anti-virulence genes.

### Probiotic Compositions:

The probiotic compositions include compositions comprising the probiotic bacteria, optionally with an appropriate carrier or stabilizer. The probiotic compositions may include different dosages of the bacterium.

In some embodiments, the probiotic compositions include compositions consisting essentially of the probiotic bacteria. The probiotic compositions may include different dosages of the bacterium.

In some embodiments, the probiotic compositions comprise dead and/or inactive probiotic bacteria.

In some embodiments, the probiotic compositions comprise heat-killed probiotic bacteria.

In some embodiments, the probiotic composition further comprises at least one other probiotic bacteria and/or yeast, optionally selected from Escherichia coli Nissle 1917, Leuconostoc mesenteroides, Lactobacillus plantarum, Pediococcus pentosaceus, Lactobacillus brevis, Leuconostoc citreum, Leuconostoc argentinum, Lactobacillus paraplantarum, Lactobacillus coryniformis, Weissella spp., Weissella spp., Leuconostoc mesenteroides, Lactobacillus fermentum, Lactobacillus acidophilus, Bifidobacterium bifidum, Streptococcus thermophilus, Lactobacillus delbrueckii subsp. bulgaricus, Lactobacillus helveticus, Lactobacillus kefiranofaciens, Lactococcus lactis, Lactococcus lactis, Gluconacetobacter xylinus, Zygosaccharomyces sp., Acetobacter pasteurianus, Acetobacter aceti, Saccharomyces boulardii and Gluconobacter oxydans

In some embodiments, the probiotic composition further comprises lactic acid bacteria selected from the group consisting of Lactobacillus, Bifidobacterium, Streptococcus and Lactococcus or combinations thereof. Optionally, the probiotic compositions include one or more probiotic yeast strains.

In some embodiment, the probiotic composition further comprises a prebiotic. The prebiotics are carbohydrates which are generally indigestible by a host animal and are selectively fermented or metabolized by bacteria. The prebiotics include oligosaccharides such as fructooligosaccharides (FOS) (including inulin), galactooligosaccharides (GOS), trans-galactooligosaccharides, xylooligosaccharides (XOS), chitooligosaccharides (COS), soy oligosaccharides (e.g., stachyose and raffinose) gentiooligosaccharides, isomaltooligosaccharides, mannooligosaccharides, maltooligosaccharides and mannanoligosaccharides. Optionally, the combined probiotic and prebiotic is a symbiotic.

The probiotic compositions may be provided as a dried, powdered or lyophilized form.

The probiotic compositions may be provided as a solid oral form. Solid forms include tablets, capsules, pills, troches or lozenges, cachets, pellets, powders, or granules or incorporation of the material into particulate preparations of polymeric compounds such as polylactic acid, polyglycolic acid, etc. or into liposomes.

The probiotic compositions may be provided in liquid form including emulsions, solutions, suspensions, and syrups.

The probiotic compositions may include other components such as inert diluents; carriers, adjuvants, wetting agents, emulsifying and suspending agents; and sweetening, flavoring, and perfuming agents.

The probiotic compositions include nutritional compositions, i.e., food products that comprise the probiotic bacteria alone or in combination with other probiotic bacteria. The food product can be a dairy product, for example, milk or a milk-based product. Exemplary milk sources include, without limitation, cattle, sheep, goat, yak, water buffalo, horse, donkey, reindeer and camel.

The milk can be whole milk or milk that has been processed to remove some or all of the butterfat, e.g., 2% milk, 1% milk or no-fat milk. In some embodiments, the milk can be previously pasteurized and or homogenized, dried and reconstituted, condensed or evaporated. Fractions of milk products including casein, whey protein or lactose may also be used.

The food product can be a cereal product, for example, rice, wheat, oats, barley, corn, rye, sorghum, millet, or triticale. The cereal product can be a whole grain or be milled into a flour. The food product can be a single kind of cereal or a mixture of two or more kinds of cereals, e.g., oat flour plus malted barley flour. The cereal products can be of a grade and type suitable for human consumption or can be products suitable for consumption by domestic animals.

The food product can also be a vegetable or a fruit product, for example, a juice, a puree, a concentrate, a paste, a sauce, a pickle or a ketchup. Exemplary vegetables and fruits include, without limitation, squashes, e.g., zucchini, yellow squash, winter squash, pumpkin; potatoes, asparagus, broccoli, Brussels sprouts, beans, e.g., green beans, wax beans, lima beans, fava beans, soy beans, cabbage, carrots, cauliflower, cucumbers, kohlrabi, leeks, scallions, onions, sugar peas, English peas, peppers, turnips, rutabagas, tomatoes, apples, pears, peaches, plums, strawberries, raspberries, blackberries, blueberries, lingonberries, boysenberries, gooseberries, grapes, currants, oranges, lemons, grapefruit, bananas, mangos, kiwi fruit, and carambola.

The food product can also be a “milk” made from grains (barley, oat or spelt “milk”) tree nuts (almond, cashew, coconut, hazelnut or walnut “milk”), legumes (soy, peanut, pea or lupin “milk”) or seeds (quinoa, sesame seed or sunflower seed “milk”).

Also contemplated are food products comprising animal proteins, for example, meat, for example, sausages, dried meats, fish and dried fish products and/or convenience foods.

### Methods of Use

The disclosed probiotic bacteria and compositions comprising the same can be used, for example, to promote gut health including improving intestinal barrier function and to maintain or regulate intestinal homeostasis.

In some embodiments, the disclosed probiotic bacteria and compositions comprising the same are useful in the treatment or prevention of gastrointestinal disorders or diseases including antibiotic-associated diarrhea, infectious childhood diarrhea, ulcerative colitis, IBS, IBD, and antibiotic-associated microbial dysbiosis.

In some embodiments, the disclosed probiotic bacteria and compositions comprising the same can be used to treat or prevent gastrointestinal tract infection including recurrent C. difficile infection, Salmonella serotypes infection and enterohemorrhagic E. coli infection

In some embodiments, the disclosed probiotic bacteria and compositions comprising the same can be used to improve gut immune function.

In some embodiments, the disclosed probiotic bacteria and compositions comprising the same can be used to increase the number the release of antimicrobial peptides.

To gain a better understanding of the invention described herein, the following examples are set forth. It should be understood that these examples are for illustrative purposes only. Therefore, they should not limit the scope of this invention in any way.

### EXAMPLE 1

### Characterization of Probiotic Bacterium

Rouxiella badensis—Like Probiotic Bacteria and Medium:

The Rouxiella badensis—like probiotic bacteria was isolated from the blueberry microbiota deposited with the ATCC acting in its capacity as an International Depository Authority on Jul. 22, 2020 under Accession Number PTA-126681 and referred to Rouxiella badensis acadiensis. The Rouxiella badensis acadiensis was growth in Tripticase Soy Agar (TSA) (Britania, Buenos Aires, Argentina) and Potato Dextrose Agar (PDA) (Britania, Buenos Aires, Argentina) at 30° C. and 37° C. for 24 h.

### Bacterial Identification:

DNA extracted from a pure culture of the RBL probiotic bacteria was sequenced using Pacbio RS Il technology. The sequencing produced three contigs having a length of 4,929,777 bp, 120,600 bp and 82,542 bp with a % GC content of 53.0%, 49.4% and 44.1% respectively.

The two smaller contigs representing potentially two native plasmids were identified. The two smaller contigs contain several genes involved in conjugal transfer.

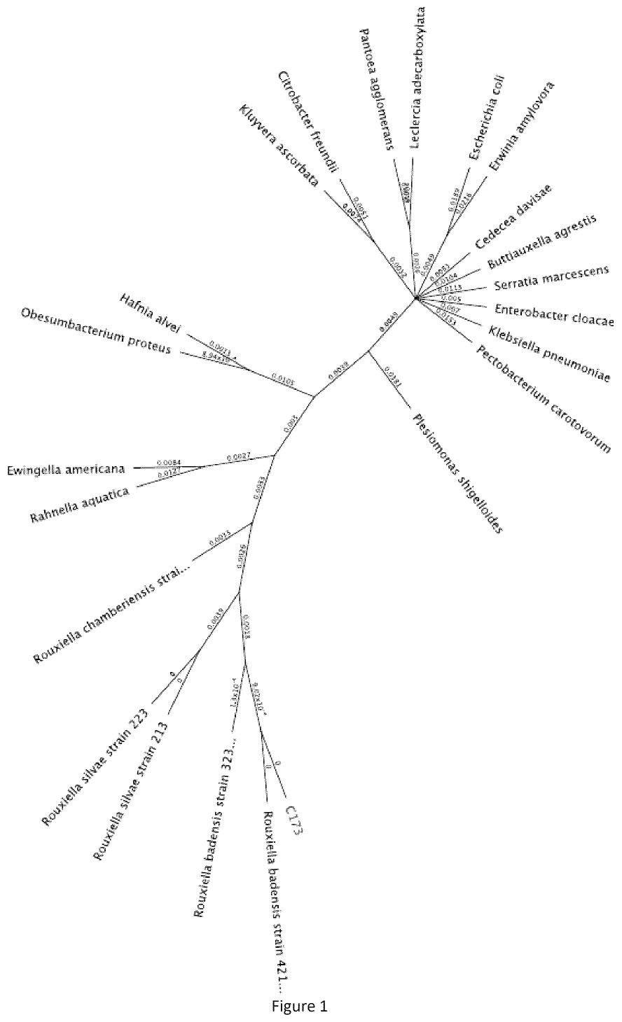

The sequence of the rrs gene (16S rDNA) from the Pacbio sequencing of the RBL probiotic bacteria was aligned (Geneious alignment) to the same rrs genes found in Le Flèche-Matéos J Syst Evol Microbiol. 2017 May;67 (5): 1255-1259. All sequences were trimmed to 1357 bp. Referring to FIG. 1, a genetic tree was generated using the neighbour-joining algorithm, and bootstrap analysis was performed with 1000 replicates. RBL probiotic bacteria rrs gene shows perfect sequence homology to the Rouxiella badensis strain 421 (100% identity) which suggest that the RBL probiotic bacteria is part of the Rouxiella genus.

In order to distinguish the species, two other gene sequences that show increased evolutionary heterogeneity (groL and gyrB) and are therefore better to discriminate among species and subspecies were assessed. The sequences of the RBL probiotic bacteria groL and gyrB genes was compared to the sequences of the three Rouxiella strains for which the whole genome sequences are available. The table below details the identity similarity between the RBL probiotic bacteria and the known Rouxiella strains for rrs, groL and gyrB.

| | | % Identity | % Identity | % Identity |
|---|---|---|---|---|
| Bacteria | Strain | rrs | groL | gyrB |
| Rouxiella badensis | 323 | 99.9% | 99.9% | 99.8% |
| Rouxiella badensis | 421 | 100% | n/a | n/a |
| Rouxiella silvae | 213 | 99.3% | 93.6% | 88.2% |
| Rouxiella silvae | 223 | 99.3% | n/a | n/a |
| Rouxiella chamberiensis | 130333 | 99.4% | 92.3% | 90.1% |

The RBL probiotic bacteria showed extremely high homology with both strains of Rouxiella badensis. As the genome of Rouxiella badensis strain 421 is not available in the public database, the only genes assessed where rrs, fusA, pyrG, rplB, rpoB and sucA.

Partial sequences of five housekeeping genes of Rouxiella badensis strain 421 (fusA, pyrG, rplB, rpoB and sucA) were available to perform a multi-locus sequence analysis (MLSA) (Le Flèche-Matéos 2017, Le Flèche-Matéos Int J Syst Evol Microbiol. 2015 June;65 (Pt 6): 1812-8.). These partial sequences of these genes from Rouxiella badensis strain 421 were used to search the genome of the RBL probiotic bacteria as well as the other Rouxiella strains, plus selected, fully sequenced, bacterial chromosomes. The five partial sequences were concatenated, in alphabetical order, for each bacterium, and a genetic tree created from the alignment was done as described above.

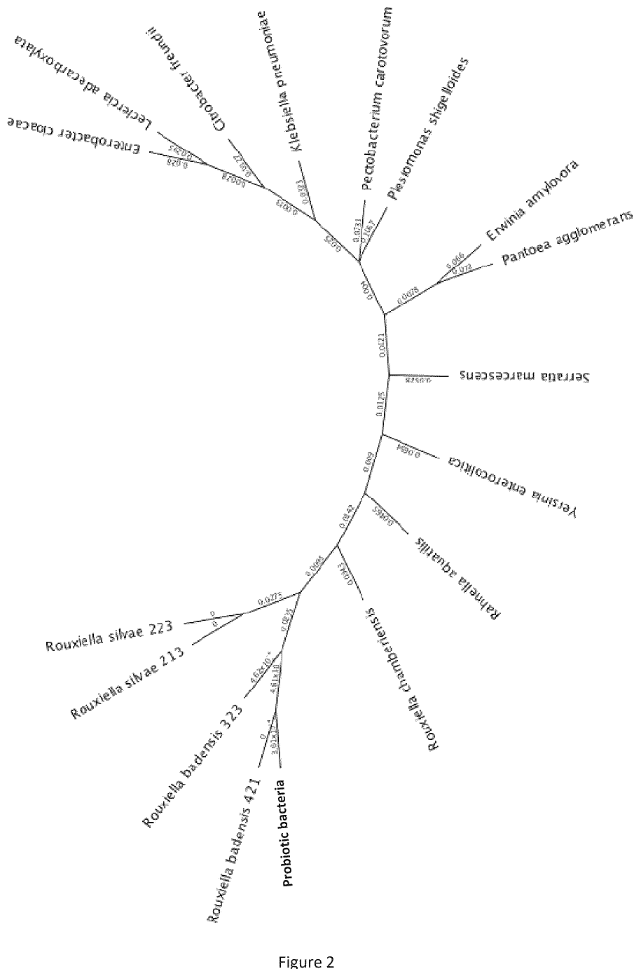

Referring to FIG. 2, the tree generated by MLSA mirrored the results obtained using rrs gene analysis, Consistent with the RBL probiotic bacteria being part of the Rouxiella genus. To determine more closely the level of homology between the Rouxiella strains and the RBL probiotic bacteria, especially the two R. badensis, each of the five sequences used in MLSA was analyzed individually (table below).

| | | % Identity | % Identity | % Identity | % Identity | % Identity |
|---|---|---|---|---|---|---|
| Bacteria | Strain | fusA | pyrG | rplB | rpoB | sucA |
| R. badensis | 323 | 100% | 100% | 100% | 99.8% | 99.8% |
| R. badensis | 421 | 100% | 100% | 100% | 100% | 99.8% |
| R. silvae | 213 | 96.2% | 97.4% | 98.5% | 95.3% | 89.0% |
| R. silvae | 223 | 96.2% | 97.4% | 98.5% | 95.3% | 89.0% |
| R. chamberiensis | 130333 | 93.7% | 95.4% | 97.9% | 94.1% | 88.0% |

For the five genes, as well as for rrs, the closest match to the genes of the RBL probiotic bacteria are the genes from Rouxiella badensis strain 421, followed by Rouxiella badensis strain 323. Comparison of common sequence fragments between the RBL probiotic bacteria and the two strains of Rouxiella badensis are consistent with RBL probiotic bacteria belonging to the Rouxiella genus and more specifically being highly related to the badensis species. The specific strain was named Rouxiella badensis acadiensis.

### Sequences:

(rrs)
SEQ ID NO: 1
ttttaattgaagagtttgatcatggctcagattgaacgctggcggcaggcctaacacatgcaagtcgagcggtagcacggg
agagcttgctctctgggtgacgagcggcggacgggtgagtaatgtctgggaaactgcctgatggagggggataactactg
gaaacggtagctaataccgcatgatgtcgcaagaccaaagtgggggaccttcgggcctcacgccatcggatgtgccca
gatgggattagctagtaggtggggtaatggctcacctaggcgacgatccctagctggtctgagaggatgaccagccaca
ctggaactgagacacggtccagactcctacgggaggcagcagtggggaatattgcacaatgggcgcaagcctgatgca
gccatgccgcgtgtgtgaagaaggccttagggttgtaaagcactttcagcgaggaggaaggcgttgtagttaatagctgca
acgattgacgttactcgcagaagaagcaccggctaactccgtgccagcagccgcggtaatacggagggtgcaagcgtt
aatcggaattactgggcgtaaagcgcacgcaggcggtttgttaagtcagatgtgaaatccccgagcttaacttgggaactg
catttgaaactggcaagctagagtcttgtagaggggggtagaattccaggtgtagcggtgaaatgcgtagagatctggag
gaataccggtggcgaaggcggccccctggacaaagactgacgctcaggtgcgaaagcgtggggagcaaacaggatt
agataccctggtagtccacgctgtaaacgatgtcgacttggaggttgtgcccttgaggcgtggcttccggagctaacgcgtt
aagtcgaccgcctggggagtacggccgcaaggttaaaactcaaatgaattgacgggggcccgcacaagcggtggagc
atgtggtttaattcgatgcaacgcgaagaaccttacctactcttgacatccagagaatttgctagagatagcttagtgccttcg
ggaactctgagacaggtgctgcatggctgtcgtcagctcgtgttgtgaaatgttgggttaagtcccgcaacgagcgcaacc
cttatcctttgttgccagcacgtaatggtgggaactcaaaggagactgccggtgataaaccggaggaaggtggggatgac
gtcaagtcatcatggcccttacgagtagggctacacacgtgctacaatggcgtatacaaagagaagcgaactcgcgaga
gcaagcggacctcataaagtacgtcgtagtccggattggagtctgcaactcgactccatgaagtcggaatcgctagtaatc
gtagatcagaatgctacggtgaatacgttcccgggccttgtacacaccgcccgtcacaccatgggagtgggttgcaaaag
aagtaggtagcttaaccttcgggagggcgcttaccactttgtgattcatgactggggtgaagtcgtaacaaggtaaccgtag
gggaacctgcggttggatcacctccttacc
(groEL)
SEQ ID NO: 2
atgtcagctaaagatgtaaaattcggtaacgacgcccgcgtaaaaatgctgaacggcgttaacattctggctaacgcagtt
aaagtgaccttaggccctaaaggccgtaacgtcgttttggacaaatcttttggtgcgccaacgattactaaagacggcgtttc
tgttgcgcgtgaaatcgaactggaagacaagttcgaaaacatgggcgctcagatggtgaaagaagttgcgtccaaagct
aacgatgctgcgggtgacggtactacgactgcaaccgtgctggctcaggccatcatcaccgaaggtctgaaagctgttgc
tgccggtatgaacccaatggacgttaagcgcggtatcgacaaagctgtcgttaacgcggttgaagagctgaaaaagctgt
ccgttccttgctctgactctaaagctatcgctcaggttggtactatttctgccaactccgacgaaaccgtgggcaaactgattg
ctcaggcgatggaaaaagtaggcaaagaaggcgttatcaccgttgaagaaggcactggcctgatcgacgagctggac
gttgttgaaggtatgcagtttgaccgcggctacctgtctccttacttcatcaacaagccagaaactggcgctgttgaacttgaa
agcccgttcatcctgctggccgacaaaaaaatctccaacattcgcgaaatgctgccagttctggaagccgttgctaaagcc
ggtaaaccactgcttatcgttgcagaagacgttgaaggtgaagcactggcaacgctggttgttaacaccatgcgtggcatc
gtgaaagttgctgctgttaaagcaccaggcttcggcgaccgtcgtaaagctatgctgcaagacatcgcaaccctgaccgg
cggtaccgtaatctctgaagagatcggtatggagctggaaaaagcgaccctggaagacatgggtcaggctaagcgcgtt
gttatcaacaaagacaccaccatcatcatcgatggtatcggtgacgaaaccaccattcagggccgcgtaaatcagatcc
gtcagcagatcgaagaagcgacctctgactacgataaagaaaaactgcaagagcgtgtggctaaactggcaggcggc
gttgccgttctgaaagtgggcgcagcgactgaagttgaaatgaaagagaagaaagcacgcgttgaagatgccctgcac
gctacccgtgctgcggttgaagaaggcgtggttgccggtggtggtgttgcgctgatccgcgttgctcacaaactggctggcc
tgaaaggcgacaacgaagaccagaccgtaggtatcaaagttgcactgcgtgcaatggaagcacctctgcgtcagatcgt
tatcaacgccggtgaagaagcttctgttatcgccaacaacgtgaaagcgggtgaaggcagctacggctataacgcttaca
ccgaagaatacggcgatatgatcgcgatgggtatcctggatccaactaaagttacccgttctgctctgcagtacgcgtcttct
gtagctggcctgatgatcaccaccgagtgcatgattaccgacctgcctaaagacgacaaagtcgacatgggcggcgctg
gcggtatgggtggaatgggcggcatgggcggcatgatgtaa
(gyrB)
SEQ ID NO: 3
atgtcgaattcttatgactcctcaagtatcaaggtattaaaagggctggacgcggtgcgtaagcgccccggcatgtatatcg
gcgataccgatgacggcactggtctgcaccacatggtattcgaggttgtggacaacgctatcgacgaagccctcgctggc
cactgtaaagagattctggttacgattcatgcggataactccgtctccgttcaggatgatggtcgtggcattccaaccggcatt
cacgaagaagaaggcatctctgccgcagaagtcatcatgactgttcttcacgccggcggtaaatttgacgataactcatac
aaagtgtccggcggcctgcacggcgtgggtgtatctgttgttaacgccctgtctgaaaagctggagttggttatccgtcgtga
aggtaaagttcacgaacaaacctacgtccacggcgagcctcagaatccactgaaagtcgttggcgagaccgaactaac
cggaacaaccgtacgcttctggccgagcttccagaccttcacaaaccataccgaattcgagtacgacatcctggccaag
cgcctgcgcgagctgtcgttcctgaactccggcgtggctatccgcctgcacgacaagcgtgatgacaaacaggatcactt
ccactacgagggcggcattaaggcgttcgtggaatacctgaacaaaaacaagaatccaatccacccgaccgtgttctatt
tctctaccatgaaagacgacatcggcgtggaaattgctctgcaatggaacgacggtttccaggaaaacatttactgctttac
caacaatattccacagcgtgacggcgggactcacctcgccggtttccgtgcggcgatgacccgtaccctgaacgcgtac
atggataaagaaggctacagcaagaaatccaaaatcagtgccaccggtgacgacgcccgtgaaggcctgatcgccgtt
gtttctgtaaaagtgccggatcctaagttctcttctcagaccaaagacaagctggtgtcgtcagaagtgaaatcagcggttg
aaaccctgatgaacgaaaagctggttgattatctgatggaaaacccgggcgacgcgaaaatcgttgtcggcaaaatcat
cgatgcggctcgtgcgcgtgaagcggcacgtaaagcccgtgaaatgacgcgtcgtaagggcgcgttggatttggccggt
ctgccgggcaaattggctgactgtcaggaacgcgatccggctcactccgaactgtacctggtggaaggggactcggcgg
gcggatctgcgaagcagggccgtaaccgcaagaatcaggccattttgccgctgaaaggtaaaatccttaacgtcgagaa
agcgcgtttcgataaaatgctctcttctcaggaagtcgccacgttgattacggcgttgggctgcggcattggccgtgacgaat
acaacccggacaaactgcgctatcacagcatcatcatcatgaccgatgccgacgtcgatggttcacacatccgaactctg
ctgctgaccttcttctatcgtcagatgcctgaaatcgttgagcgcggccacatctttatcgctcagccgccgctgtacaaggtg
aaaaaaggtaagcaggaacagtacattaaagatgacgaagcgatggatcagtatcaaatcgccatcgcaatggacgg
cgcaacgctgcacactaacgccagcgcacctgccttggccggtgagccgctggagaagctggttgccgagcattacgc
ggtacagaaaatgattggtcgtatggagcgtcgctacccgcgtgcgctgttgaacaatctggtttatcagccaacgctgga
agaaacttctctgaccgatcgtgaaaaagtgcagcagtggatggaagaactggtcgtgttgcttaacgaaaaagagtctc
acggcagcagctacagcgctatcgtgcgtgaaaatcgtgagcgtcagatctacgaaccggttctgcgtatccgtactcac
ggcgttgatactgattatgacctcgattttgatttcatccacggtggtgaataccgtaagctttgtgctctgggtcagcaacttcg
cggcctgatcgaagaagatgcctacatcgagcgcggcgaacgccgtcagccgatttccaccttcgagcaagcgctgga
atggctggtgaaagagtcccgtcgcggtctgtccgtacagcgttataaaggtctgggcgaaatgaacccagaacagctgt
gggaaacgaccatggatcctgatcagcgtcgcatgctgcgcgtgaccatcaaagatgctatcgctgccgatcagctgttc
accacgctgatgggagacgccgttgaaccacgccgcgctttcatcgaagaaaacgccctgaaagcagcgaatatcgat
atctaa
(fusA)
SEQ ID NO: 4
ggtgtaaaccacaagatcggtgaagtacacgacggcgcagcaaccatggactggatggagcaggagcaggaacgtg
gtattaccatcacgtctgctgcgactacctgttactggtctggtatggctaagcagttcgaaccacaccacatcaacatcatc
gacaccccaggacacgttgacttcaccatcgaagttgaacgttctatgcgtgttcttgatggcgcagtaatggtttactgtgct
gttggcggcgttcagccacagtctgaaaccgtatggcgtcaagctaacaaatataaagttcctcgtatcgcgttcgttaaca
aaatggaccgtatgggtgctaacttcctgaaagttgttggtcagatcgaacagcgtctgaatgcaactccggttcctctgcag
ctggctatcggcgcagaagagaaattcaccggcgttgttgaccttgtcaagatgaaagcaatcaactggaacgaagaag
atcagggcgtgaccttcgaatacgaagagatcccagctgatatgcaagaactggctgaagaatggcgccagaaactgg
ttgaagccgctgctgaaggttctgacgagctgatggagaaattctttggtggcgaagagctgaccgaag
(pyrG)
SEQ ID NO: 5
attctcgaagcccgtggtttgaacgttaccatcatgaaactcgatccgtacatcaacgtcgatccgggtaccatgagcccaa
cccagcatggtgaagtcttcgtgaccgaagatggggctgaaacagatctcgatctgggtcactacgaacgtttcattcgca
ctaaaatgactcgccgtaacaactttactaccggtcgtatttactccgacgtcctgcgcaaagagcgccgtggcgattatctc
ggcgcgaccattcaggttatcccgcacatcaccaacgcaatcaaagagcgcatcattgaa
(rplB)
SEQ ID NO: 6
cgttacatcctggcgccgaaaggcctgaaagtgggtgaccagatccaatctggcgttgatgctgcaatcaaagtgggtaa
cacccttccgatgcgcaacatcccagtaggttcaacggttcacaacgtagaaatgaaaccaggtaaaggcggccagct
ggctcgttctgctggtgcctacgttcagatcgttgctcgtgatggttcctacgtaactctgcgtctgcgctctggcgaaatgcgt
aaagttcagatcgactgccgcgccaccatgggtgaagttggtaactctgaacatatgcttcgcgttctgggtaaagcaggt
gctgcacgt
(rpoB)
SEQ ID NO: 7
cgtctgtctctgggcgacctcgacaccctgatgcctcaggacatgatcaacgccaagccaatttcggcggcggtgaaaga
gttcttcggctcaagccagctttcacaatttatggaccagaacaacccgttgtccgagataacgcacaagcgtcgtatctctg
cattgggcccgggcggtttgacccgtgaacgtgctggctttgaagttcgagacgtacacccgactcactacggtcgcgtat
gtccaatcgaaacgccagaaggtccaaacatcggtctgatcaactccttgtctgtttatgcacagaccaacgagtatggttt
cctcgaaacgccttaccgccgcgtgcgtgatggcatcgttaccgatgaaattaactatctgtctgctattgaagaaggcaac
ttcgttatcgcccaggcgaactccaacctggacgaagaaggccgcttcattgaagacctggttacctgtcgtagcaaagg
cgaatcaagcctgtttagccgcgaccaggttgactatatggacgtatctacccagcaggttgtatccgttggtgcttcactgat
tccattcctggaacacgatgacgccaaccgtgcattgatgggtgcgaacatgcaacgtcaggctgtaccaactctgcgtgc
tgacaagccgttggtgggtaccggtatggaacgtgctgtagcggttgactccggcgttaccgccgttgctaaacgtggcggt
attgttcagtacgtggatgcatcccgtatcgtaatccgtgttaacgaagacgagatgtacccgggcgaagcaggtatcgac
atttataacctgaccaaatacacccgttctaaccagaacacctgcatcaaccagatgccgtgtgtgaatctgggtgagcca
atcgagcgcggcgacgtgctggcagatggcccatcaacagacctgggcgagc
(sucA)
SEQ ID NO: 8
aagaccttcctcaaagaattaaccgcagccgaagggctggagcgctacctcggtgctaaattcccgggcgcgaaacgct
tctcgctggaaggcggcgatgccctggtacctatgcttaaggacatgatccgccacgccggtaaaaacggcacgcgtga
agtcgttctgggcatggcgcaccgcggtcgtctgaacgtattgatcaacgtgctgggtaaaaaaccccaagacctgtttgat
gaattctcaggtaagcataaagagcatctgggtaccggcgatgtgaaatatcacatgggctattcgtccgacgtcgaaact
gaaggtggtatggttcacctggcgctggcgtttaacccgtcgcaccttgaaatcgtcagcccagtggttatcggttccgttcgt
gcccgtcgcgatcgtcttgacgaagcgcgcagtaacatggttctgccaatcaccattcacggcgatgcggcgattaccggt
cagggcgtcgttcag

Rouxiella badensis acadiensis highly related to but different than Rouxiella badensis strain 323.

Rouxiella badensis strain 323 whole-genome sequencing produced 72 contigs from 417 bp to 280,267 bp. These contigs were first compared to the two plasmid sequences found in Rouxiella badensis acadiensis. No contig matching the first plasmid (120,600 bp) except for a small contig that aligned imperfectly to a repetitive gene sequence, called transposase that is often repeated several times, in bacterial genome and plasmids was identified. The same analysis was done on Plasmid 2, and no contig from R. badensis was found matching this sequence from Rouxiella badensis acadiensis strain. The results are consistent with the two plasmids found in Rouxiella badensis acadiensis being absent from R. badensis strain 323.

To further compare the two strains, the 72 R. badensis contigs were aligned on the chromosome of Rouxiella badensis acadiensis strain. Two contigs from R. badensis didn't match to Rouxiella badensis acadiensis genome: contig 38 and 62.

Contig 62 is a small contig of 4.5 kb and has the highest homology (73%) to a region of the Serratia sp. P2ACOL2 genome. It contains a primase, three hypothetical genes, a transcriptional regulator and an Ash-like/host cell division inhibitor Icd-like protein.

Contig 38 contains several phage related genes. When blasted against the Genbank database, this contig shows homology to a Pantoea stewartia plasmid, that was identified as a linear phage plasmid (Duong 2018). Consequently, this contig was analyzed using the phage identifying tool PHASTER (phaster.ca). The results indicated that this sequence is related to a Klebsiella phage, and it contains all the genes necessary for the phage.

The genome and two plasmids from Rouxiella badensis acadiensis were then analyzed using PHASTER to detect the presence of phage sequences. Neither plasmid 1 or plasmid 2 contain a potential intact phage.

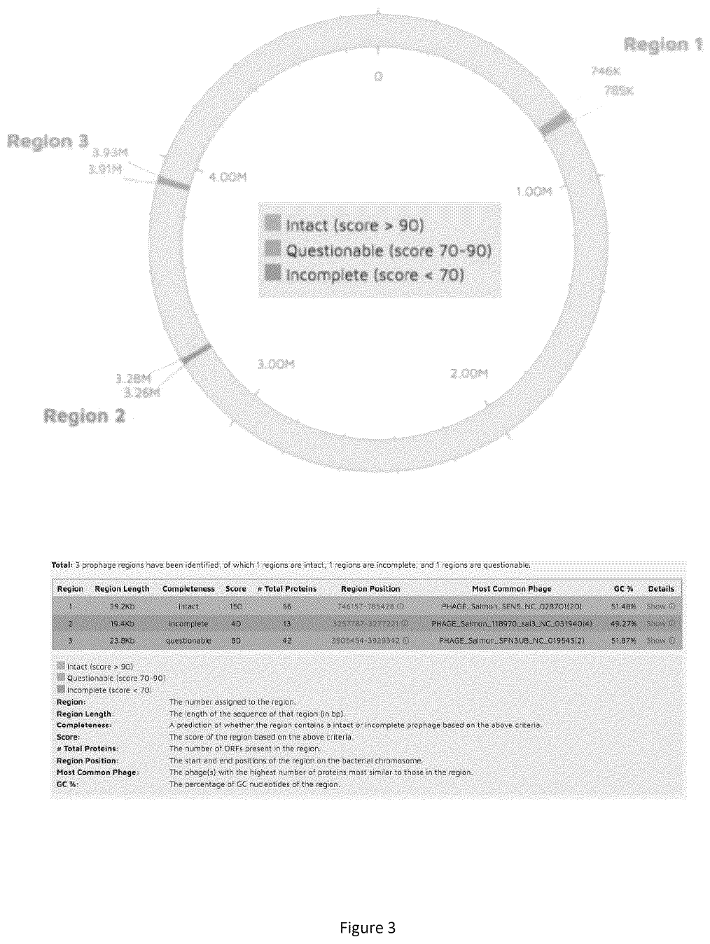

For the chromosome, FIG. 3 shows the three regions identified as prophage regions, with one being an intact prophage. This intact prophage is similar, but not identical, to the temperate Salmonella SN5 phage. The SN5 phage was found in Salmonella enterica subsp. salamae and was active against five pathogenic Salmonella enterica sbsp. enterica (Mikalová 2017).

The contigs from R. badensis were also used to perform a LASTZ genome alignment with the Rouxiella badensis acadiensis chromosome. The result indicates that the two genomes share 99.5% pairwise identity covering 93.3% of the Rouxiella badensis acadiensis genome.

A quick single nucleotide polymorphism (SNP) analysis from the LASTZ alignment detects 16,867 SNPs, with 4,349 of them being non-synonymous SNPs inside genes. The Pacbio sequencing showed that the colicin V production protein has a stop codon in the middle of the gene of the Rouxiella badensis acadiensis. The other proteins associated with “Bacteriocins and ribosomally synthesized antibacterial peptides” category that was listed in the 2015 sequencing report are all present and intact in R. badensis and Rouxiella badensis acadiensis.

A difference in phenotype traits between R. badensis and Rouxiella badensis acadiensis was observed. As shown in the table below, the Rouxiella badensis acadiensis doesn't produce acid from L-rhamnose, while the other strain can. This is consistent with the finding that both the L-rhamnose transporter (rhaT) and the L-rhamnulokinase (rhaB) have a gene mutation creating frameshifts, and consequently the creation of stop codons.

| | | R. badensis | R. badensis |
|---|---|---|---|
| Acid formed from: | C173 | 323 | 421 |
| N-Acetyl-glucosamine | − | − | − |
| D-Arabitol | − | + | − |
| D-Fucose | − | + | + |
| Glycerol | − | + | + |
| D-Lyxose | − | − | − |
| D-Maltose | − | + | + |
| D-Melezitose | − | + | + |
| Methyl-D-glucose | − | − | − |
| D-Raffinose | + | + | + |
| L-Rhamnose | − | + | + |
| D-Sucrose | + | + | + |
| D-Trehalose | + | − | + |
| D-Turanose | − | + | + |
| D-Xylitol | − | − | − |

The Rouxiella badensis acadiensis Produces Colonies with Different Morphologies

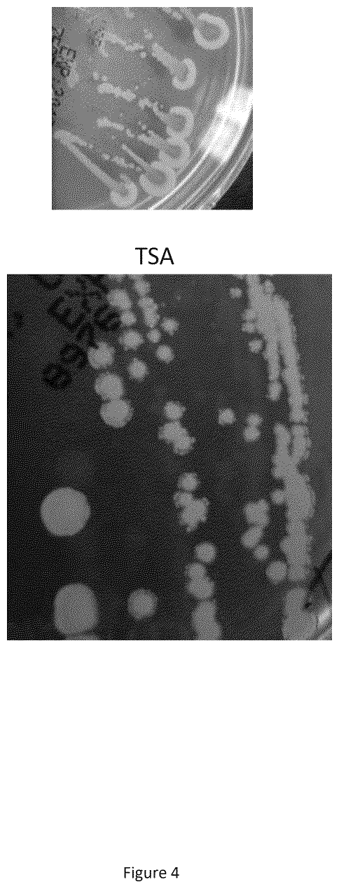

Referring to FIG. 4, pure cultures of the Rouxiella badensis acadiensis as confirmed by sequencing produced different colony morphologies.

Rouxiella badensis acadiensis were Tolerant to Acid Medium

200 μl aliquots of an overnight culture of Rouxiella badensis acadiensis were added to 5 ml of Tripticase Soy Broth previously adjusted with hydrochloric acid at pH: 2, 3, 4, 5 and 7. After incubation at 30° C. for 2 and 4 h, the cultures were plated on TSA and incubated for an additionally 72 h at 30° C. These times were set based on there are the maximum times in which the strain could be in the stomach. Colony counts were performed at the end of the incubation period (22). Results were expressed as Log₁₀ of the CFU/ml.

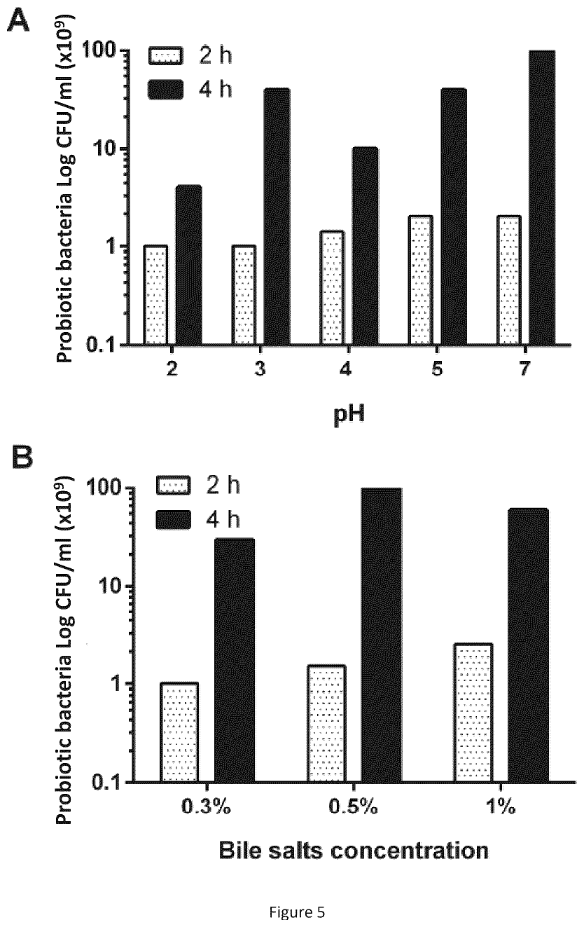

Referring to FIG. 5A, the Rouxiella badensis acadiensis grew at the low pH assayed.

Rouxiella badensis acadiensis were Bile Salt Resistance

Resistant to growth in different concentrations of bile salt was assayed. 200 μl aliquots of an overnight culture of Rouxiella badensis acadiensis was added to 5 ml of Tripticase Soy Broth containing different concentrations of bile salt: 0.3, 0.5 and 1%. After incubation at 30° C. for 2 and 4 h, each culture was serially diluted, spread in TS agar plates, and incubated at 30° C. for 72 h, followed by determination of CFU count. Results were expressed as Log₁₀ of the CFU/ml.

Referring to FIG. 5B, similar bacteria counts in agar plate at the different conditions of bile salts assayed was observed showing that the Rouxiella badensis acadiensis is resistant to high bile salt concentration.

Rouxiella badensis acadiensis did not Harm Human Erythrocytes

The hemolytic activity of the Rouxiella badensis acadiensis was evaluated. Briefly, aliquots of 10⁹ CFU/ml Rouxiella badensis acadiensis was plated on blood agar base containing 5% fresh blood, and incubated at 30° C. for 24, 48 and 72 hours. The presence of hemolysis was read at the different times assayed. Results indicate that the Rouxiella badensis acadiensis did not harm human erythrocytes. In particular, the Rouxiella badensis acadiensis did not display any α- or β-hemolysis at 24, 48 or 72 hours of incubation at 30° C.

Rouxiella badensis acadiensis did not Impact Macrophage Viability

Peritoneal macrophages removed aseptically from Balb/c mice were employed to determine the viability by the 3-(4,5-dimethylthiazol-2yl)-2,5-diphenyltetrazolium bromide (MTT) method. Macrophages (5×10⁵) were settled in a flat-bottom 96-well microplate and cultured in either the absence or the presence of increasing concentrations of the bacterium (10⁸ to 10¹⁰ CFU/ml) and a supernatant of Rouxiella badensis acadiensis culture. Plates were incubated at 37° C. in a 5% CO₂ atmosphere for 24 h. The purple formazan crystals formed were dissolved with 100 μL of DMSO and the absorbance was read at 570 nm in a microplate reader. Results were calculated as the ratio between the optical density in the presence and absence of the bacterium multiplied by 100.

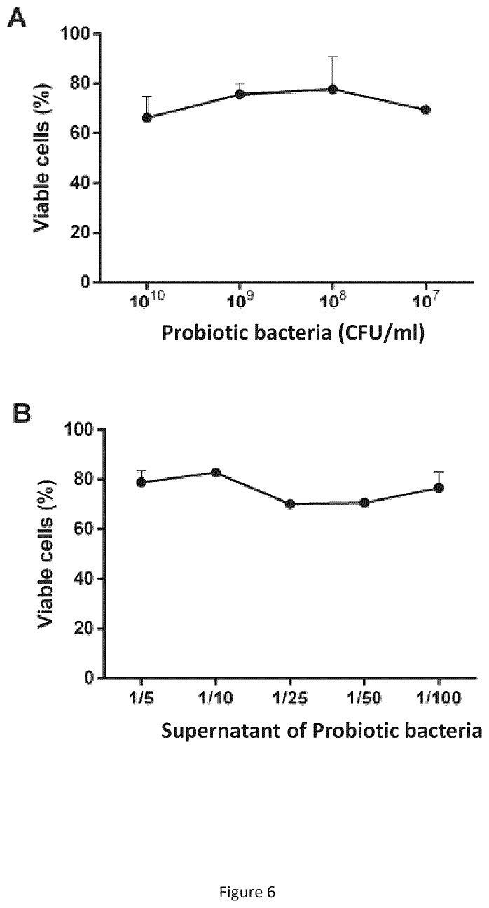

Referring to FIG. 6A, no change in macrophages viability was observed when they were expose to increasing concentration of the bacterium (10⁷ to 10¹⁰ CFU/ml). Moreover, referring to FIG. 6B, the metabolites shed to the medium during Rouxiella badensis acadiensis growth

### Example 2

### Characterization of In Vivo Effect of Rouxiella badensis acadiensis

Rouxiella badensis acadiensis did not Significant Alter the Host Intestinal Homeostasis.
Animals and diet supplementation:

BALB/c mice were provided for CERELA (San Miguel de Tucumán, Argentina) from a closed random bred colony. Animals were maintained in a room with a 12-h light/dark cycle at 22±2° C. and fed ad-libitum with conventional balanced food commercial.

Rouxiella badensis acadiensis overnight cultures were grown at 30° C. in 5 ml of sterile TSB. The cells were harvested by centrifugation at 5000 g for 10 min, washed three times with phosphate saline solution (PBS) and resuspended in 5 ml of sterile 10% (wt/vol) non-fat milk. Bacterial suspensions were diluted 1:30 in water and administered ad-libitum to the mice. The final concentration of bacteria was 2±1×10⁹ CFU/ml. These counts were periodically controlled at the beginning of the administration and each 24 h of dilution in water to avoid modifications of more than one logarithmic unit. BALB/c mice received conventional diet (Normal Control) or 10⁹ CFU/ml of Rouxiella badensis acadiensis in the drinking water during 7 days. This time corresponds to the time required for an optimal activation of the intestinal immune system for the probiotic L. casei CRL431.

For continuous supplementation with Rouxiella badensis acadiensis, 10⁹ CFU/ml of the bacterium were administered. At the end of the experimental period, intestinal microbiota, bacterial translocation, peritoneal macrophages activity and histology of the small intestine were analyzed.

The adherence capacity of Rouxiella badensis acadiensis to the epithelium was evaluated by electronic microscopy.

### Analysis of the Intestinal Microbiota:

The impact of the probiotic administration with or without prebiotic on bacterial composition at pylum level (10 most abundant phyla) in 6-8 weeks old healthy female BALB/C mice was further assessed. After one week acclimatization, mice were categorized in 4 groups as follows (6 mice in each group) (1) probiotic group, receiving 10⁹ CFU per day in drinking water; (2) prebiotic group, receiving prebiotic Protocatechuic acid (PCA), 100 mg/kg body weight, in drinking water; (3) probiotic and prebiotic group, receiving a mixture of 10⁹ CFU probiotic and PCA in drinking water and (4) control group, receiving regular drinking water.

For probiotic solution preparation, overnight cultures were grown at 30° C. in 5 mL of sterile TSB. The cells were harvested by centrifugation at 5000 g for 10 min, washed three times with phosphate-buffered saline solution (PBS) and suspended in 5 mL of sterile 10% (wt/vol) non-fat milk. Bacterial suspensions were diluted 1:30 in water and administered ad-libitum to the mice. The duration of nutritional intervention was 3 weeks. Each day, mice were provided with new probiotic or prebiotic solution at the final volume of 30 mL. At the end of intervention (day 21), mice were euthanized Metagenomic analysis were performed on cecum content, flash frozen and stored at −80° C. The metagenomics analysis was run by a shallow shotgun techniques.

Permutational multivariate analysis variation was used to estimate the effect of experimental factor on taxonomic profiles. When the effect on the prebiotic and probiotic presence as separated factors was evaluated it was found that the presence of probiotic significantly change the communities' taxonomic profiles.

| | Df | SumOfSqs | R2 | F | Pr..F. |
|---|---|---|---|---|---|
| Probiotic | 1 | 0.046 | 0.029 | 0.661 | 0.621 |
| Prebiotic | 1 | 0.218 | 0.134 | 3.097 | 0.020 |
| Probiotic:Prebiotic | 1 | 0.027 | 0.017 | 0.383 | 0.847 |
| Residual | 19 | 1.336 | 0.821 | NA | NA |
| Total | 22 | 1.627 | 1.000 | NA | NA |

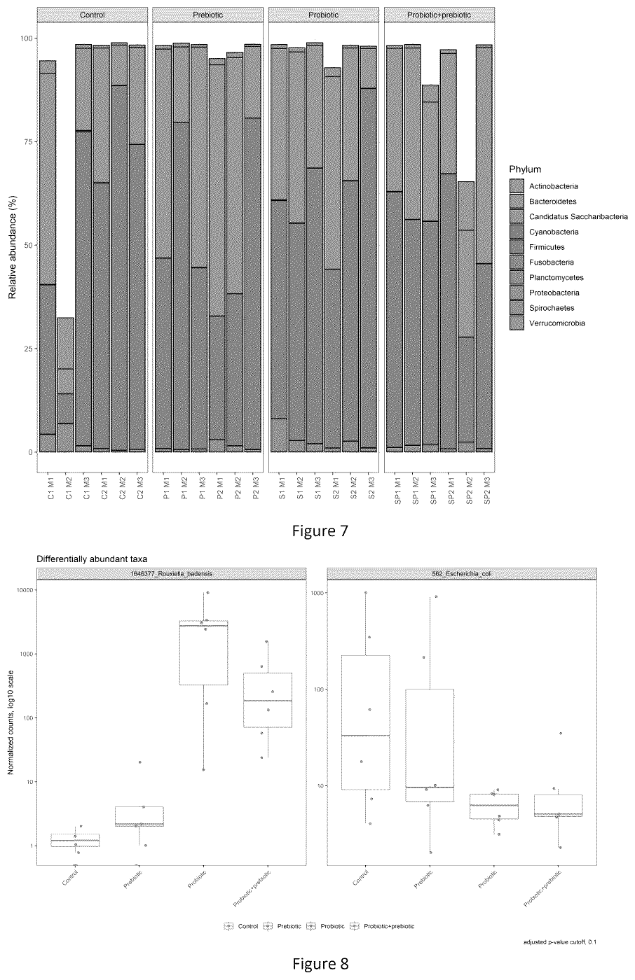

Referring to FIG. 7, the probiotic induced positive compositional changes at phyla, genus, or species level. The normal gut microbiota consists predominantly of two main phyla that includes bacteroides and firmicutes. Bacteroides have a mainly positive role. The intake of probiotic alone or in combination with prebiotic resulted in fewer firmicutes and higher bacteroidetes. The lower microbiota diversity and higher firmicutes level is linked with many disease states, including metabolic syndrome. In particular, a substantial imbalance is observed across four major bacterial phyla including Firmicutes, Bacteroidetes, Proteobacteria and Actinobacteria in Intestinal Inflammatory Bowel (IBD) disease that includes ulcerative colitis and (CD) and Crhon's disease (CD). In the results, the presence of probiotic significantly changes the communities' taxonomic profiles.

A negative binomial models (DESEq2 R package) for differential abundance testing of taxonomic and subsystem level 3 features was used. Changes due to group differences using a likelihood ratio test compared all the groups at once. Two taxa with significantly different abundance across groups were found.

| | | | | Probiotic. | |
|---|---|---|---|---|---|
| | Control | Prebiotic | Probiotic | prebiotic | pvalue |
| Rouxiella badensis | 1.060598 | 4.905935 | 3093.534128 | 459.25919 | 0.00e+00 |
| Escherichia coli | 283.971880 | 192.272673 | 6.392056 | 10.35632 | 2.26e−05 |

Referring to FIG. 8, two taxa with significantly different abundance across groups were found and linked to the administration of probiotic or SV. SV increased significantly the presence of Rouxiella badensis taxon and significantly decreased Escherichia coli.

Pathogenic Escherichia coli is associated with gut dysbiosis and has been linked to Crohn's disease (CD) patients, ulcerative colitis (UC). It was suggested that E. coli strains play a facilitative role during Intestinal Bowel Disease (IBD) flares and its pathogenesis. Controlling E. coli in this disease is considered a hallmark in preventing recurrence and treatment as well of IBD.

Clinical data reported that Gram-probiotic E coli Nissle 1917 is the only probiotic showing efficacy and safety in maintaining remission equivalent to the gold standard mesalazine in patients with ulcerative colitis.

The data support the use of the probiotic in treatment and prevention of inflammatory disease including Inflammatory Bowel Disease.

No Weakening of the Intestinal Barrier Observed in Rouxiella badensis acadiensis Feed Mice.

Weakening of the intestinal barrier allows for translocation of resident intestinal bacteria to distant sites including the spleen and liver. To assess the impact of the Rouxiella badensis acadiensis on intestinal barrier integrity, the livers and spleens from the mice described above were aseptically removed, weighed and placed into sterile tubes containing 5 ml of peptone water (0.1%). The samples were immediately homogenized and serial dilutions were made and spread onto the surface of MacConkey agar or MRS agar to assess for the presence of enterobacteria and lactobacilli in the organs. The plates were then aerobically incubated at 37° C. for 24 h. No bacterial translocation from the intestinal microbiota to the liver or the spleen was observed, suggesting long term consumption of the Rouxiella badensis acadiensis does not induce inflammatory damage capable to reducing the integrity of the intestinal barrier.

The Rouxiella badensis acadiensis did not Adhere to the Intestinal Epithelium.

The adherence of the Rouxiella badensis acadiensis to the mice epithelium was analyzed in intestinal sections taken at 5 and 15 minutes after oral administration of the bacterium. BABL/c mice received orally by gavage 100 μl of Rouxiella badensis acadiensis (10⁹ CFU/ml). Animals were sacrificed 5 and 15 minutes later. The small intestines of each mouse were removed, washed with 3 ml of PBS and 0.5 mm segments of tissue fixed in 2.66% formaldehyde, 1.66% glutaraldehyde, sodium phosphate buffer 0.1 M pH 7.4 and incubated overnight at 4° C. The samples were processed and observed with a Zeiss EM109 (Carl Zeiss NTS GmbH, Oberkochen, Germany) and Zeiss SUPRA 55-VP for transmissions and scanning electron microscopy studies, respectively.

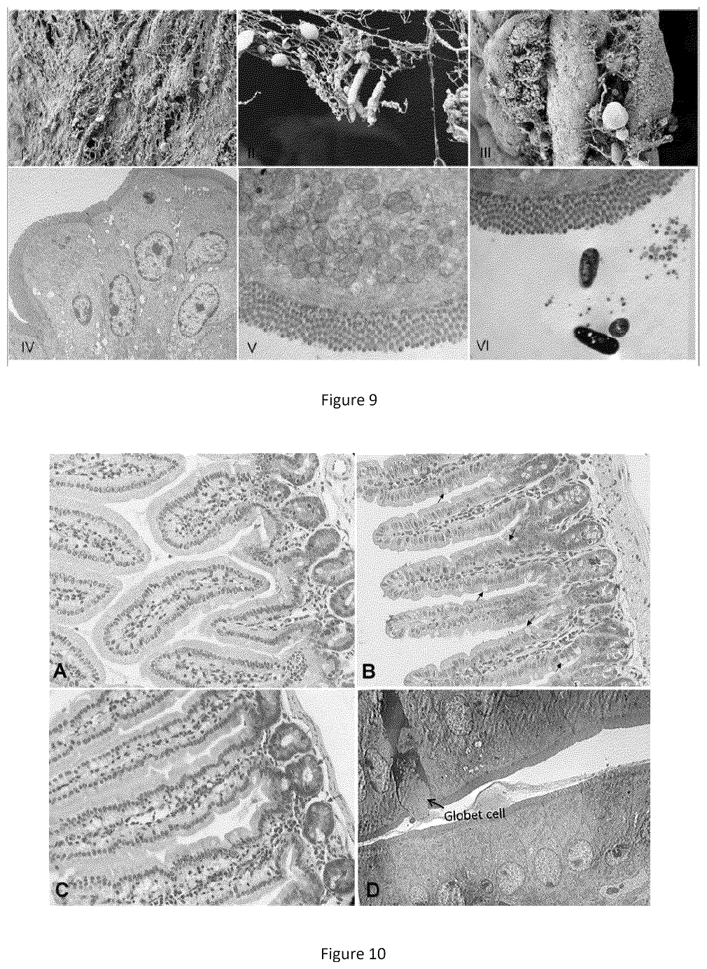

Referring to FIG. 9, by scanning and transmission electronic microscopy, no adhesion of the Rouxiella badensis acadiensis to the epithelial cell at any 5 and 15 minutes was observed.

Rouxiella badensis acadiensis Reinforced the Intestinal Epithelial Barrier without Disturbing the Intestinal Homeostasis.

The impact of the oral consumption of Rouxiella badensis acadiensis on small intestine architecture was assessed. Animals were fed with convention diet or Rouxiella badensis acadiensis (10⁹) for consecutive 7 or 90 days, respectively. Referring to FIG. 10, on hematoxylin and eosin stained tissue no inflammatory foci that could be due to the bacterial ingestion were observed. This non-inflammatory effect was also observed ex vivo. Intestinal epithelial cells from mice fed with Rouxiella badensis acadiensis did not secrete significant levels of IL-6 (63.13±16.83 and 64.32±16.31 pg/ml, for 7 and 60 days, respectively) nor IFN-γ (95.62±0.28 and 99.76±3.17 pg/ml, for 7 and 60 days, respectively) regarding control animals (IL-6:46.12±0.60 and 44.32±4.79; IFN-γ: 90.06±2.17 and 123.10±26.02 pg/ml for 7 and 90 days, respectively). These results were in agreement with the fact that the bacterium does not interact with the intestinal epithelial cells, as normal microbiota does.

An increase in the Goblet and Paneth cells in Rouxiella badensis acadiensis fed animals was observed in the hematoxylin and eosin compared to animals receiving a conventional diet. Considering these cells are responsible for mucus and antimicrobial peptides production, these results suggest that the Rouxiella badensis acadiensis reinforces natural intestine microbial barriers.

Effect of Probiotic Rouxiella badensis acadiensis on Intestinal Integrity and Prevention of LPS Induced-Inflammation

The effect of probiotic Rouxiella badensis acadiensis on intestinal integrity and prevention of LPS induced inflammation was assayed. Briefly, three weeks old female mice were used in the current study. After one-week acclimatization, mice were divided into two groups: 1—receiving 10⁹ CFU per day probiotic Rouxiella badensis acadiensis (Canan SV 53) refereeing thereafter in the legend as SV in drinking water; or 2—receiving regular water. The duration of nutritional intervention was two weeks, started from four weeks old of age until puberty (six weeks old of age). To assay the acute effect of LPS on the intestine, after two weeks of intervention, each group was divided into two groups. Half of the mice in each group were injected by LPS and half of them were injected with saline 8 hours prior to being euthanized. Therefore, the four groups were (1) Saline group (control), (2) LPS group, (3) SV+Saline group and (4) SV+LPS group.

Eight hours after LPS or saline injection, mice were euthanized and the required sample, including intestine were collected.

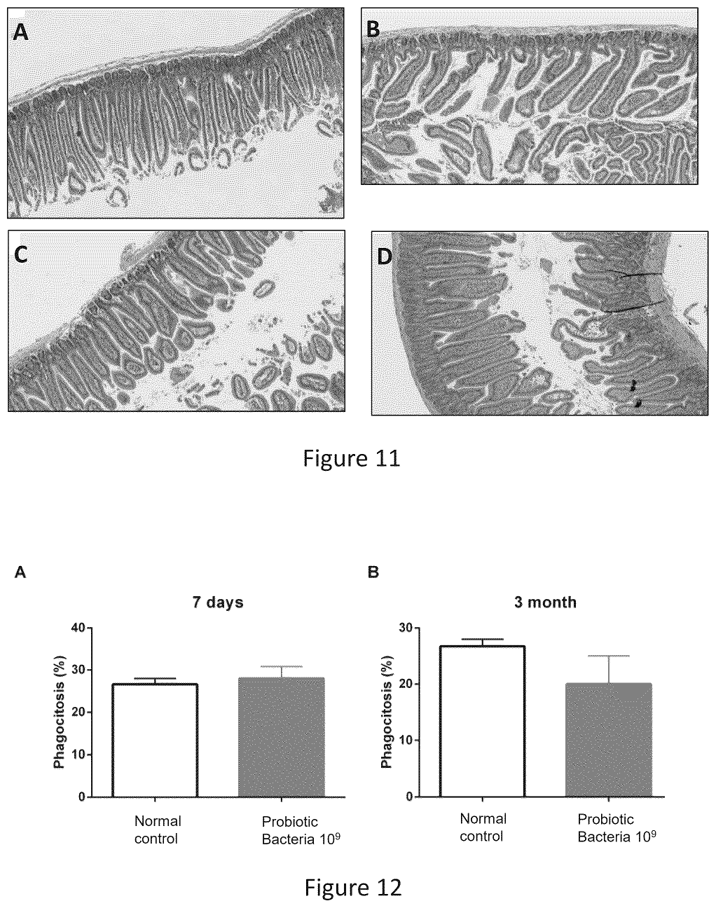

To assay the detrimental effect of LPS on intestine and to assay the protective effect of SV against LPS-induced inflammation, hematoxylin and eosin-stained slides of intestinal tissues were provided. FIG. 11 shows the differences in villi structure between groups. Blood was also collected from mice. Samples from ileum were processed and analyzed on samples tissues were sliced in 4 μm thick cuts, and stained with hematoxylin-eosin.

LPS clearly induced inflammatory changes at the intestinal villi affecting structure and high of the villi as illustrated by comparing FIG. 11A and 11B. The protective effect of the probiotic on the structure of intestine tissue is evident when FIG. 11B is compared to FIG. 11D. No morphological changes in the intestine of mice pre-treated with probiotic in response to LPS injection. The probiotic therefore provided protection against LPS-induced inflammation at the intestinal level.

As shown in FIG. 11, LPS-induced inflammation resulted in significant morphological changes in villi and epithelial structure at the intestine level. However, 2 weeks of orally administered probiotic prior to the LPS challenge was shown to have protective effects against the damaging effects of LPS by preservation of the integrity of the intestinal structure. Blood from these mice was collected. Orally administered probiotic prior to LPS challenged showed significant inhibitory effects on IL6 and TNF-α in female mice. These cytokines alter intestinal permeability through its effect on tight junction structure between epithelial cells

The probiotic prevents intestinal barrier disruption and inhibits LPS-induced inflammation suggesting the probiotic is a potential therapeutic agent against IBD and intestinal inflammation.

Supplementation with Rouxiella badensis acadiensis did not Impact Phagocytic Activity of Peritoneal Macrophages.

After Rouxiella badensis acadiensis (10⁹ CFU/ml) administration for 7 days or 3 months animals were sacrificed. Peritoneal macrophages were extracted from peritoneal cavity with 5 ml of sterile PBS, pH 7.4. Then, the cells were harvested by centrifugation at 800-1000 g for 15 min at 4° C. The resulting pellets were gently mixed with 2 ml of sterile red blood cell lysing buffer (Sigma, St Louise, USA) during 2 min. The haemolysis was stopped with PBS. The samples were again centrifuged and resuspended in RPMI-1640 medium (Sigma, St. Louis, USA) containing foetal bovine serum (FBS). Phagocytosis assay was performed using a suspension of 10⁷ Saccharomyces cerevisiae/ml. Yeast opsonized in mouse autologous serum (10%) was added to 200 μl of macrophages (10⁶ cells/ml). The mixture was incubated for 30 min, at 37° C. The percentage of phagocytosis was expressed as the percentage of phagocyting macrophages in 100 cells count using an optical microscope. This assay was also performed in mice that consume Rouxiella badensis acadiensis (10⁹ CFU/ml) for 90 consecutive days.

Referring to FIG. 12, no increase in the opsono-phagocytosis of yeast by macrophages from animals that ingested Rouxiella badensis acadiensis compared to those registered in animals that received a conventional diet was observed.

Antimicrobial Activity in the Intestinal Fluid Observed in Rouxiella badensis acadiensis Fed Mice.

The antimicrobial activity of the intestinal fluids of control animals and those fed with Rouxiella badensis acadiensis was assayed. Briefly, the small intestines of mice were removed and their content collected in sterile tube by passage 0.5 ml of 10 mM sodium phosphate buffer, pH 7.4 along the intestine. Supernatant were then collected after centrifuge at 1300×g 4° C. 15 min.

Exponential growth phase suspensions of S. Typhimurium and S. aureus adjusted at 5×10⁶ CFU in 20 μl were incubated for 2 h at 37° C. in the presence or absence of 100 μl of the intestinal fluids obtained from the different mice. Each incubation mixtures were serially diluted, spread in duplicate selective agar plates, and incubated at 37° C. for 18 h, followed by determination of CFU counts. Results were expressed as the CFU/ml of the pathogens after their incubation with the intestinal fluids.

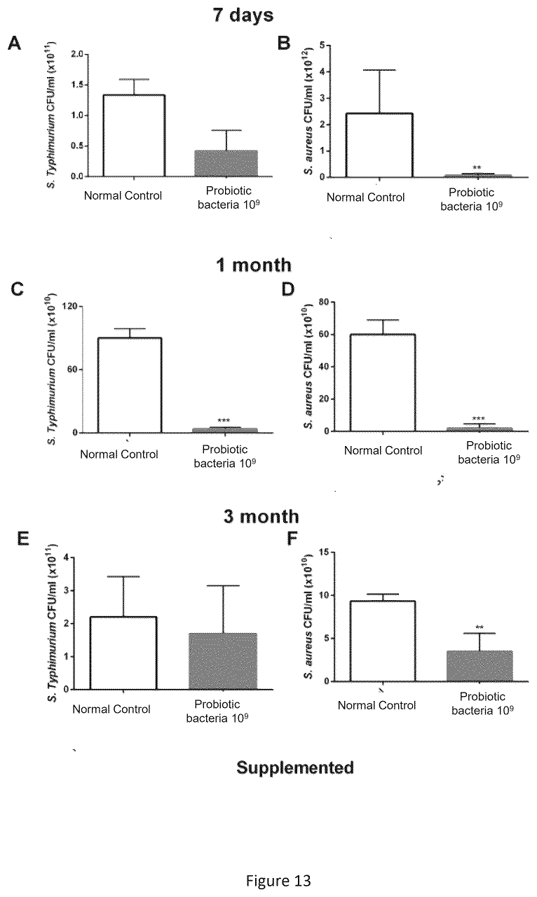

Referring to FIG. 13, after 7 days, 1 and 3 months of consecutive Rouxiella badensis acadiensis feeding, samples of intestinal fluids were taken and assayed against pathogenic bacteria. A decrease in the CFU/ml of S. typhimurium and S. aureus were observed in the presence of intestinal fluids of animals fed with Rouxiella badensis acadiensis compared to intestinal fluid from control mice.

Rouxiella badensis acadiensis Administration had No Significant Impact on the Body Weight.

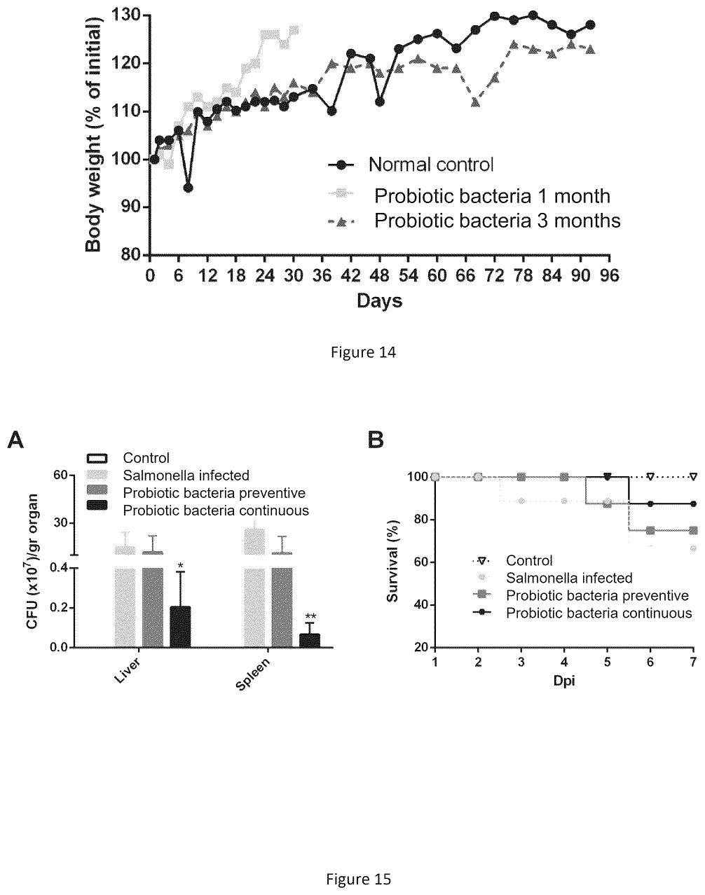

As some Rouxiella badensis acadiensis ingested for long period of times induce a weight lost, the impact of Rouxiella badensis acadiensis on body weight was assessed. Referring to FIG. 14, a slight increase in the body weight of mice that ingested the bacterium for 1 months was observed. However, no significant changes in the body weight was observed in animals that received Rouxiella badensis acadiensis for 3 months.

Continuous Administration of Rouxiella badensis acadiensis Protected against Salmonella enterica Serovar Typhimurium Infection.

Referring to FIG. 15, groups of BALB/c mice weighing 26±4 g were used in the study were as follow: G-1 animals that received a conventional diet (Control); G-2 mice upon a conventional diet challenged by intragastric inoculation with 1×10⁷ CFU/m of Salmonella enterica serovar Typhimurium (Salmonella infected); G-3: animals fed with Rouxiella badensis acadiensis (10⁹ CFU/ml) the 7 days previous to Salmonella challenge (Rouxiella badensis acadiensis preventive); and G-4 animals fed with Rouxiella badensis acadiensis (10⁹ CFU/ml) the 7 days previous to the challenge and continuous with the ingestion of Rouxiella badensis acadiensis the 7 days after Salmonella challenge. Animals were sacrificed 7 days post infection.

The liver and spleen were aseptically removed, weighed and placed into sterile tube containing 5 ml of peptone water (0.1%). The samples were homogenized and serial dilutions were spread onto the surface of McConkey agar. The number of CFU was determined after aerobically incubation for 24 h at 37° C. Results were expressed as CFU/g of organ.

The S-IgA antibodies in the intestinal fluid of the small intestine were measured by ELISA 7 days post challenge. The procedure used for the specific anti-Salmonella IgA antibodies was carried out as described previously by Leblanc et al 2004 (29), using goat anti-mouse IgA (alpha-chain-specific) conjugated peroxidase. The optical density was as measured at 450 nm using a VERSA Max Microplate reader (Molecular devices, Sunnyvale, CA, USA).

For the specific anti-Salmonella S-IgA antibodies determinations, plates were coated with 50 μl of a suspension of heat-inactivated S. Typhimurium (1010 CFU/ml) and incubated overnight at 4° C. Nonspecific protein-binding sites were blocked with PBS containing 0.5% nonfat milk. The samples from the intestinal fluid of mice were diluted in 0.5% nonfat milk in PBS and then incubated at room temperature for 2 h. After washing with PBS containing 0.05% Tween 20, the plates were incubated 1 h with peroxidase-conjugated anti-IgA-specific antibodies. Plates were again washed and the tetramethylbenzidine (TMB) reagent was added. The reaction was stopped with H2SO4 (2 N). The absorbance was read at 450 nm. Results are expressed as concentration (μg/ml) of IgA in the intestinal fluid.

The increase in the number of Paneth cells and in the in vitro antimicrobial activity in the intestine of animals fed with Rouxiella badensis acadiensis led as to investigate in vivo whether the bacterium can protect against S. typhimuriun infection. Mice that received Rouxiella badensis acadiensis seven days previous to the Salmonella challenge and continue with the ingestion of Serratia the days after the infection, showed a better survival than Salmonella infected animals. Additionally, when we analyzed the translocation of the bacteria to others organs, we observed a decrease in CFU/ml in those animals, regarding the infected ones in both liver and spleen (p<0.05). These results suggest that Rouxiella badensis acadiensis administered by oral route reinforce the intestinal barrier through an increase in antimicrobial peptides production that protects against Salmonella infection. By contrast the consumption of Rouxiella badensis acadiensis just the days previous of Salmonella infection was not enough to protect against Salmonella challenge.

Total and specific anti-Salmonella s-IgA in preventive or continuous administration, did not increase in mice given Rouxiella badensis acadiensis regarding to infected mice receiving conventional diet. The values obtained were: specific anti-Salmonella s-IgA 0.6256±0.05665 and 0.6559±0.09763, for infected control and Rouxiella badensis acadiensis group respectively, and 10.24±1.260 and 9.798±1.246 μg/ml for total s-IgA, respectively.

### The Profile of Cytokines and Immunoglobulins Induced at the Intestinal Mucosa, in the Intestinal Fluid and Serum of Balb/C Mice of Rouxiella Badensis Acadiensis Feed Mice

To evaluate the effect of the novel probiotic on modulating different cytokines in the serum and at the intestinal level, 8-week-old Balb/c female mice weighing 20-25 g received by gavage 10⁸ CFU of Rouxiella badensis acadiensis/mouse for 7 consecutive days provided in 1% sucrose dissolved in 1×PBS pH7.4. Control mice received the same volume of 1% sucrose in 1×PBS instead. All mice received simultaneously a conventional balanced diet ad libitum and water. Test and control animals were sacrificed after 7 days of probiotic administration.

In vivo cytokines were determined in serum and intestinal fluid by ELISA. The number of IgA and IgG producing (IgA+ and IgG+) cells in the lamina propria of the small intestine of mice that received Rouxiella badensis acadiensis was determined on histological slices from the ileum by a direct immunofluorescence method using anti-mouse IgA FITC conjugate or anti-mouse IgG FITC conjugate. The results were expressed as the number of IgA+ or IgG+ cells (positive=fluorescent cell) per 10-fields (objective magnification 100×)

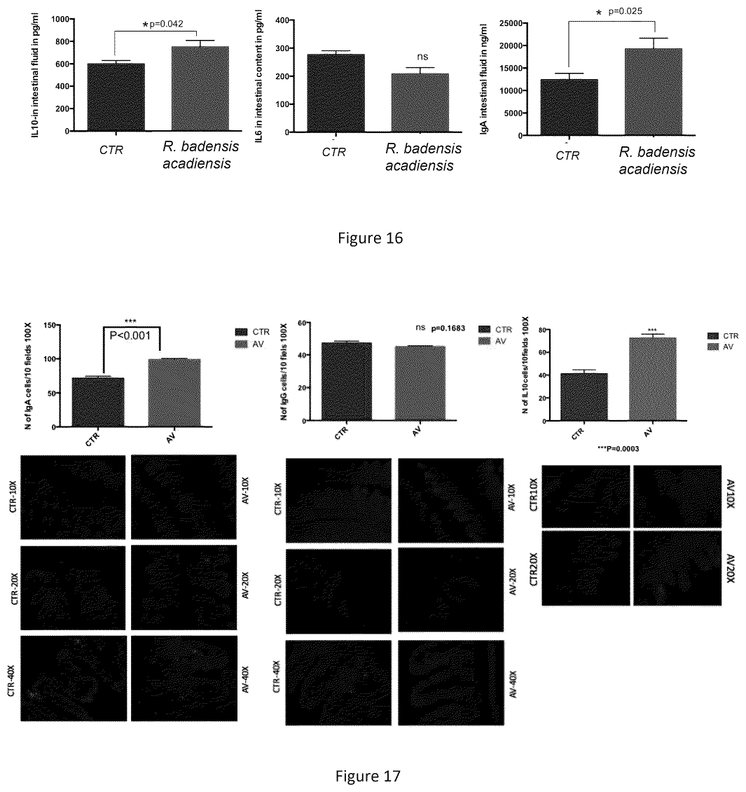

The mucosal immunomodulating capacity of Rouxiella badensis acadiensis was assessed in this study by examining its effects on the IgA and selected cytokines (pro-inflammatory cytokine IL-6 and anti-inflammatory cytokine IL-10) in both the gut mucosa and the intestinal contents. A significant increase in IL-10 was observed in the intestinal fluid of animals that received Rouxiella badensis acadiensis for 7-days when compared with control mice (FIG. 16A). There was no change in the levels of IL-6 (FIG. 16B). Secretory IgA were shown to be increased significantly in the intestinal fluid of mice fed the bacterium compared to the control group (FIG. 16C) Similarly, the number of IgA+ and IL-10+cells in the lamina propria of the ileum increased after Rouxiella badensis acadiensis administration, while the number of IgG+ cells did not change. These results confirm that no inflammatory immune response was observed by Rouxiella badensis acadiensis at concentration of 10⁸/mouse for a period of 7 days (FIG. 17).

Rouxiella badensis acadiensis Upregulated the Expression of Anti-Inflammatory miRNAs: miR146a, miR145 in the Ileum and Brain

Brains and ileum were collected from Balb/c mice fed for 7 consecutive days by 1% sucrose in 1× PBS or 10⁸ CFU Rouxiella badensis acadiensis/mouse (in 1% sucrose in 1× PBS). Tissue samples were saved in RNA later at −80° C. RNA is extracted by Qiagen miRNeasy kit using beads, aliquoted and preserved at −80° C. The expression of miR145 and miR146a was assessed by qRT-PCR.

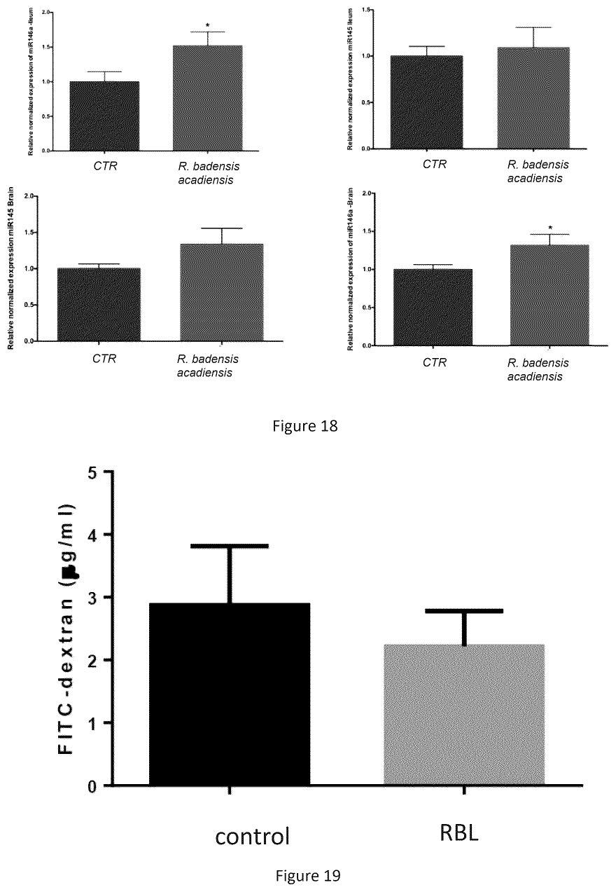

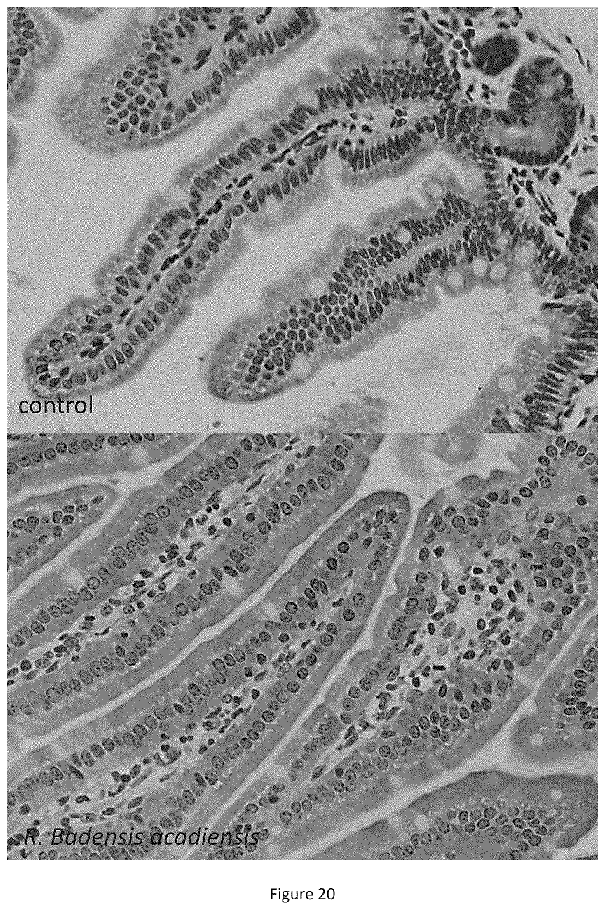

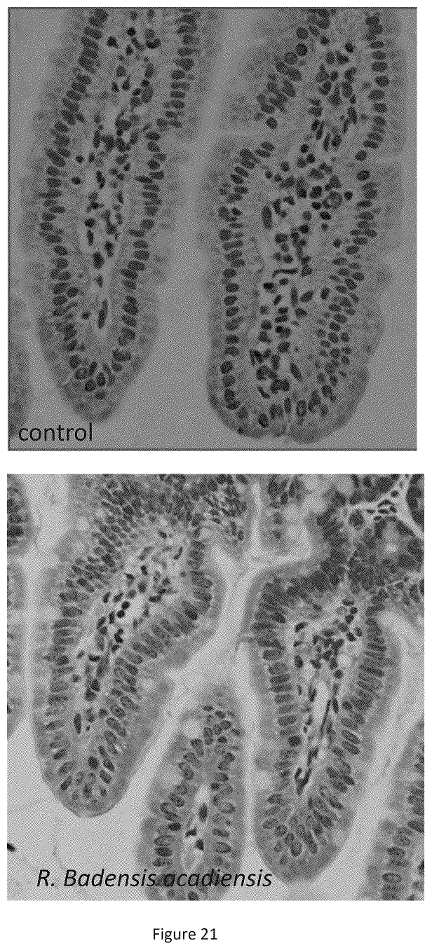

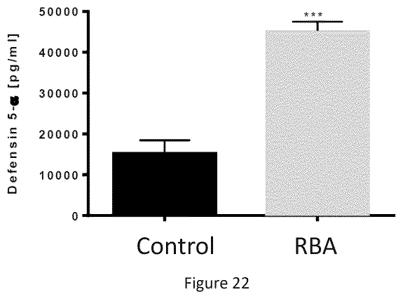

Referring to FIG. 18, consumption of Rouxiella badensis acadiensis upregulated the expression of anti-inflammatory miRNAs: miR146a, miR145 in the ileum and brain. Although no statistically significant changes were observed in the expression of miR145 in ileum or brain, a trend for an increase in the brain was observed. However, a significant increase in miR146a expression was observed in ileum and brain revealing the anti-inflammatory potential of Rouxiella badensis acadiensis.

Although the invention has been described with reference to certain specific embodiments, various modifications thereof will be apparent to those skilled in the art without departing from the spirit and scope of the invention. All such modifications as would be apparent to one skilled in the art are intended to be included within the scope of the following claims.

1-12. (canceled)

13. A method of promoting gut mucosal immunity in a mammal, the method comprising orally administering an isolated Rouxiella badensis, optionally Rouxiella badensis acadiensis.

14. A method of restoring and/or increasing an intestinal epithelial barrier, the method comprising orally administering an isolated Rouxiella badensis, optionally Rouxiella badensis acadiensis.

15. The method of claim 14, wherein the method comprises orally administering the isolated Rouxiella badensis daily for at least 7 days, at least 14 days, at least 21 days, at least 28 days, or at least 90 days.

16. The method of claim 14, wherein the method increases number of Goblet and/or Paneth cells in the small intestine epithelium.

17. A method of protecting against a Salmonella infection, the method comprising orally administering an isolated Rouxiella badensis.

18. The method of claim 17, wherein the Rouxiella badensis comprises Rouxiella badensis acadiensis.

19. The method of claim 17, wherein the method comprises orally administering the isolated Rouxiella badensis daily for at least 7 days, at least 14 days, at least 21 days, at least 28 days or at least 90 days.

20. The method of claim 15, wherein the method increases number of Goblet and/or Paneth cells in the small intestine epithelium.

21. The method of claim 18, wherein the method comprises orally administering the isolated Rouxiella badensis daily for at least 7 days, at least 14 days, at least 21 days, at least 28 days or at least 90 days.
